(19) United States
(12) Patent Application Publication
(10) Pub. No.: US 2006/0247667 A1
(43) Pub. Date: Nov. 2, 2006

(54) System and method for bonding closure of an intra-cardiac opening using energy

(76) Inventors: John E. Ahern, Morrisville, VT (US); Carol A. Devellian, Topsfield, MA (US); John A. Wright JR., Lexington, MA (US)

Correspondence Address: KIRKPATRICK & LOCKHART NICHOLSON GRAHAM LLP, STATE STREET FINANCIAL CENTER, ONE LINCOLN STREET, BOSTON, MA, 02111-2950, US

(21) Appl. No.: 11/412,626

(22) Filed: Apr. 27, 2006

Related U.S. Application Data
(60) Provisional application No. 60/675,584, filed on Apr. 28, 2005.

(51) Int. Cl. A61B 17/22

(52) U.S. Cl. 606/159

(57) ABSTRACT

The invention generally relates to systems and methods for percutaneous closure of intra-cardiac openings, such as a patent foramen ovale (PFO). In one embodiment, a closure system includes an elongated member coated with a bonding material. The bonding material adheres to the intra-cardiac opening when energy is applied to the elongated member. The system may also include an energy riser, such as a protuberance on the surface of the elongated member or a modification to the surface of the elongated member. In another embodiment, the closure device includes a catheter containing the elongated member or a locator, such as a balloon or hook. The elongated member may be inserted into an intra-cardiac opening, such as a patent foramen ovale.

### REFERENCE TO RELATED APPLICATIONS

This application claims priority to and the benefit of U.S. Provisional patent Application No. 60/675,584, filed on Apr. 28, 2005, the entire disclosure of which is incorporated by reference herein.

### TECHNICAL FIELD

The invention generally relates to devices, systems, and related methods for closing intracardiac openings. More particularly, the invention features devices, systems, and related methods for the percutaneous transluminal closure of patent foramen ovale (PFO).

### BACKGROUND

The human heart is divided into four compartments or chambers. The left and right atria are located in the upper portion of the heart and the left and right ventricles are located in the lower portion of the heart. The left and right atria are separated from each other by a muscular wall, the intra-atrial septum, and the ventricles are separated by the interventricular septum.

Either congenitally or by acquisition, abnormal openings (holes or shunts) can occur between the chambers of the heart or between the great vessels, causing inappropriate blood flow. Such deformities are usually congenital and originate during fetal life when the heart forms from a folded tube into a four chambered, two-unit, i.e., atrial and ventricular, system. The septal deformities result from the incomplete formation of the septum, or muscular wall, between the left and right chambers of the heart and can cause significant problems.

One such septal deformity or defect, a patent foramen ovale, is a persistent, usually flap-like opening in the wall between the right atrium and the left atrium of the heart. Since left atrial pressure is normally higher than right atrial pressure, the flap typically stays closed. Under certain conditions, however, right atrial pressure exceeds left atrial pressure, creating the possibility for abnormal right to left shunting of venous blood that can allow blood clots and other toxins to enter the systemic circulation. This is particularly problematic for patients who are prone to forming venous thrombus, such as those with deep vein thrombosis or clotting abnormalities.

Nonsurgical (i.e., percutaneous) closure of a patent foramen ovale and similar cardiac openings, such as an atrial septal defect or a ventricular septal defect, can be achieved using a variety of mechanical closure devices. These closure devices typically have a metallic structural framework with a scaffold material attached thereto. Many currently available closure devices, however, are often complex to manufacture, are inconsistent in performance, require a technically complex implantation procedure, lack anatomic conformability, and lead to complications (e.g., thrombus formation, chronic inflammation, residual leaks, perforations, fractures, and conduction system disturbances).

Improved devices, systems, and related methods for closing cardiac openings, such as, for example, a patent foramen ovale, are, therefore, needed.

### SUMMARY OF THE INVENTION

The present invention provides a closure system and related method for the percutaneous transluminal closure of an intracardiac opening. In one aspect, a system of the invention may include, for example, a catheter including a lumen axially disposed between a proximal end and a distal end and containing an elongated member slideably disposed in the lumen of the catheter. The elongated member has a length between a proximal end and a distal end. In one embodiment, the elongated member has a plurality of spokes at the distal end. An energy activatable bonding material is positioned on the outer surface of each of the plurality of spokes. An energy source, such as a radio frequency energy, electrical resistance, ultrasound energy, laser energy, chemical energy, microwave energy, sonic energy, or a thermal resistance heating energy source, is operatively connected to the proximal end of the elongated member. The bonding material is activated by transferring energy from the energy source to the bonding material. In one embodiment, the elongated member transfers energy from the energy source to the energy activatable bonding material.

Various embodiments of this aspect of the invention include the following features. The closure system may include bonding material in the form of a sleeve having a lumen. The sleeve is slideably disposed on the distal end of an elongated member. The bonding material may include a coating releaseably adhered to the elongated member which is releasable on application of energy. The bonding material may include bioabsorbable material. Bioabsorbable materials include bioresorbable materials such as poly-L-lactic acid, polylactic acid, or polyglycolic acid, or copolymers or combinations thereof; a biological material, such as collagen, cellulose, or animal derived tissues, e.g., the intestinal collagen layer comprising tunica submucosa of a porcine small intestine; or a synthetic material such as an absorbable or non-absorbable polymer. Absorbable synthetic material includes resorbable synthetic material. In one embodiment, the elongated member further comprises a lumen with a retractable distal stop slideably disposed in the lumen.

Moreover, in other embodiments, the system may include an energy riser, such as a protuberance on the surface of the elongated member, a noninsulated portion of the elongated member disposed between two insulated portions of the elongated member, a roughened surface of the elongated member, and a segmental alteration in the elongated member's material properties, such as by a secondary or tertiary process involving coating the material, or placing alternate materials within a segment of the elongated member to sharply change the material properties in the segment relative to the rest of the elongated member.

According to another embodiment, the energy riser may be located on the bonding material, such as a protuberance on the surface of the bonding material, a roughened surface of the bonding material, a segmental alteration in the bonding material's material properties, such as by a secondary or tertiary process involving coating the material, or placing alternate materials within a segment of the bonding material to sharply change the material properties in the segment relative to the rest of the bonding material.

In various embodiments of this aspect of the invention, the elongated member includes a noninsulated portion. The cross section of the elongated member may be noncircular.

According to additional embodiments, the invention includes a first locator distal to the energy source for positioning the elongated member in the cardiac opening. In another embodiment, the invention includes a second locator proximal to the energy source for positioning the elongated member in the cardiac opening. In another embodiment, the invention includes a first distal locator and a second proximal locator. In various embodiments of this aspect of the invention, the locator may be a balloon or a hook, and may be insulated or non-insulated.

In another aspect, the invention relates to a method for percutaneous transluminal closure of an intracardiac opening via a transvascular route, e.g., via the femoral vein, including the steps of inserting a catheter comprising an elongated member having a plurality of spokes, and a locator into a patient; locating the patent foramen ovale in the patient's heart with the locator; positioning the elongated member comprising one or more sleeves of bonding material; applying energy to the elongated member; adhering the bonding material to the intracardiac opening; and removing the elongated member and locator; removing the catheter, elongated member and locator from the patient.

The foregoing and other aspects, features, and advantages of the invention will become more apparent from the following description taken in conjunction with the accompanying drawings.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 is a cutaway view of a heart illustrating a patent foramen ovale.

FIG. 2A is a schematic perspective view of the distal portion of a closure device, including a delivery catheter and an elongated member, including a plurality of distal spokes, for the percutaneous transluminal closure of an intracardiac opening according to another illustrative embodiment of the invention.

FIG. 2B is a schematic perspective view of a closure device, including a delivery catheter, an elongated member including a plurality of distal spokes, and an energy source, for the percutaneous transluminal closure of an intracardiac opening according to an illustrative embodiment of the invention.

FIG. 3A is a schematic perspective view of a portion of a closure device according to another illustrative embodiment of the invention.

FIG. 3B is a schematic perspective view of a portion of another closure device according to another illustrative embodiment of the invention.

FIG. 4 is a schematic perspective view of a portion of a closure device having energy risers according to another illustrative embodiment of the invention.

FIG. 5 is a schematic perspective view of a portion of a closure device having energy risers according to another illustrative embodiment of the invention.

FIGS. 6A and 6B illustrate a series of steps for implanting the closure device from a top perspective schematic view according to an illustrative embodiment of the invention.

FIG. 7A is a side schematic view of a portion of a closure device including a locator positioned in a patent foramen ovale according to an illustrative embodiment of the invention.

FIG. 7B is a top schematic perspective view of a portion of the locator of FIG. 7A according to an illustrative embodiment of the invention.

FIG. 8 is a side schematic view of a portion of a balloon locator positioned in a patent foramen ovale according to another illustrative embodiment of the invention.

FIG. 9 is a schematic side view of a portion of a closure device including exemplary flexible members for positioning the closure device in an intracardiac defect, according to an illustrative embodiment of the invention.

FIG. 10A is a schematic side view of a portion of a closure device including a set of flexible members for positioning the closure device in an intracardiac defect, wherein the flexible members are partially extended from a catheter according to an illustrative embodiment of the invention.

FIG. 10B is a schematic side view of the flexible members of FIG. 10A fully extended from the opening in the catheter, according to an illustrative embodiment of the invention.

FIG. 11 is a schematic side view of a portion of a closure device including a set of flexible members for positioning the closure device in an intracardiac defect, according to an illustrative embodiment of the invention.

FIG. 12A is a schematic side view of a portion of a closure device including a flexible member for positioning the closure device in an intracardiac defect, according to an illustrative embodiment of the invention.

FIG. 12B is a schematic end-on view of the portion of a closure device including a flexible member of FIG. 12A, according to an illustrative embodiment of the invention.

FIG. 13A is a schematic side view of a portion of a closure device including spiral shaped flexible member according to an illustrative embodiment of the invention.

FIG. 13B is a schematic end-on view of the portion of a closure device including the flexible member of FIG. 13A, according to an illustrative embodiment of the invention.

FIG. 14A is a schematic side view of a portion of a closure device including flexible members collapsed within a catheter, according to an illustrative embodiment of the invention.

FIG. 14B is an illustration of the set of flexible members of FIG. 14A, in an extended configuration beyond the distal end of the catheter, according to an illustrative embodiment of the invention.

### DETAILED DESCRIPTION OF THE INVENTION

The present invention features systems and related methods for closing cardiac openings, such as, for example, the patent foramen ovale described below.

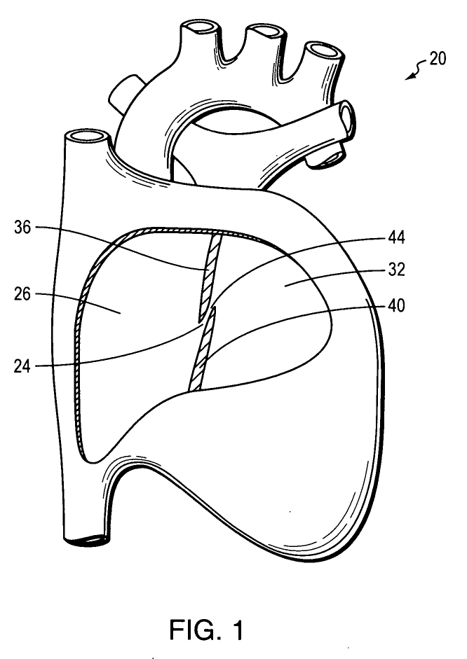

FIG. 1 depicts a cutaway view of a heart 20. The heart 20 includes a septum 24 that divides a right atrium 26 from a left atrium 32. The septum 24 includes a septum secundum 36 and a septum primum 40. An exemplary cardiac opening, a patent foramen ovale 44, that is to be corrected by the system and related method of the present invention is located between the septum secundum 36 and the septum primum 40. The patent foramen ovale 44 provides an undesirable fluid communication between the right atrium 26 and the left atrium 32 and, under certain conditions, allows for the abnormal shunting of blood and other toxins between the right atrium 26 and the left atrium 32. If the patent foramen ovale 44 is not closed or obstructed in some manner, a patient is placed at higher risk for an embolic stroke in addition to other circulatory abnormalities.

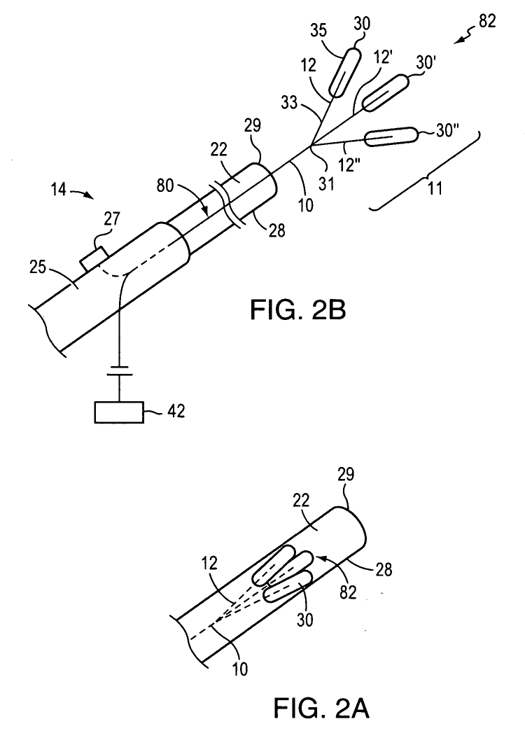

FIGS. 2A and 2B are schematic perspective views of a portion of a closure device 14, including a delivery catheter 28, an elongated member 10, and an energy source 42, for the percutaneous transluminal closure of an intracardiac opening according to an illustrative embodiment of the invention. The closure device 14, in the illustrative embodiment, for example, includes a handle 25 including an actuator 27, and a delivery catheter 28 including a lumen 22 in which the elongated member 10 is slideably disposed. The proximal end 80 of the elongated member 10 is disposed within the lumen 22 of the delivery catheter 28.

Referring to FIGS. 2A and 2B, according to one illustrative embodiment of the invention, the elongated member 10 is slideable from a first position to a second position by operator directed axial motion of the actuator 27. The actuator 27 is operatively joined to the proximal end 80 of the elongated member 10. While the delivery catheter 28 is stationary, the elongated member 10 slides from a first position, for example, as illustrated in FIG. 2A, wherein the distal end 82 of the elongated member 10 is enclosed and collapsed within the lumen 22 of the delivery catheter 28, to a second position, for example, as illustrated in FIG. 2B, wherein the distal end 82 of the elongated member 10 is in an expanded configuration beyond the outside of the lumen 22 of the delivery catheter 28. According to this illustrative embodiment, the delivery catheter 28 is stationary during the sliding movement of the elongated member 10.

Referring to FIGS. 2A and 2B, according to another illustrative embodiment of the invention, the delivery catheter 28 is slideable from a first position to a second position by operator directed axial motion of the actuator 27 while the elongated member 10 is stationary. The actuator 27 is operatively joined to the delivery catheter 28. The delivery catheter 28 slides from a first position, for example, as illustrated in FIG. 2A, wherein the distal end 82 of the elongated member 10 is enclosed by and collapsed within the lumen 22 of the delivery catheter 28, to a second position, for example, wherein the distal end 82 of the elongated member 10 is in an expanded configuration outside of the lumen 22 of the delivery catheter 28, as shown, for example, in FIG. 2B. According to this illustrative embodiment, the elongated member 10 is stationary during sliding movement of the delivery catheter 28.

The distal end 82 of the elongated member 10, for the purpose of illustrating exemplary embodiments of slideable movement illustrated in FIGS. 2A and 2B, may include, for example, a distal portion 11 of the elongated member 10 having one or more spokes 12. Each spoke 12 has a fixed end 33 and a free end 35, with the fixed end 33 being connected to the main body of the elongated member 10. In a further embodiment, bonding material 30 is attached to the elongated member 10. For example, in one embodiment, bonding material 30 is attached to one location on the elongated member 10, while in another embodiment, bonding material 30 is attached to two or more locations on the elongated member 10. In another embodiment, bonding material 30 is attached to one or more spokes 12. For example, bonding material 30 is attached to one location on one or more spokes 12 at the distal end 82 of the elongated member 10 in one embodiment, while in another embodiment, bonding material 30 is attached to two or more locations on one or more spokes 12. In one embodiment, the bonding material 30 is releaseably coupled to the elongated member 10 or spoke 12. The slideable movement of the elongated member 10 and/or the delivery catheter 28 may be directed, for example, by an actuator 27 located, for example, on the handle 25 of the closure device 14.

With continued reference to FIG. 2B, the cross-section of the elongated member 10 may include a variety of geometric configurations including round, oval, square, rectangular and flat (not shown). The distal end 82 of the elongated member 10, for example, may also include one spoke 12 or a plurality of spokes 12, 12′, 12″. The shape of the spoke 12 may include a variety of geometric configurations including straight, bent, spiral and S-shaped (not shown). Although the illustrative embodiment includes three spokes 12, 12′, 12″, it is contemplated that there may be more than three spokes, and as many as 16 spokes. Each of the illustrative spokes 12, 12′, 12″ may be arranged in the same plane. According to another embodiment, the spokes 12, 12′, 12″ may be arranged in an arc, with each spoke 12 separated from an adjacent spoke 12′, 12″ by an angle of separation of between 5 degrees and 180 degrees. Each spoke 12, 12′, 12″ includes a portion of bonding material 30, 30′, 30″, respectively, in the form of a releaseably coupled coating or a slideably disposed sleeve, described in greater detail below.

With continued reference to FIG. 2B, in one embodiment one or more of spokes 12, 12′, 12″, (generally 12), is flexibly biased relative to the other spokes 12, 12′, 12″ at the distal end 82 of the elongated member 10. Upon deployment of the elongated member 10 from the distal end 29 of the catheter 28, the spokes 12, 12′, 12″ are biased to automatically separate from one another at a pivot point 31 due to the tension forces between the spokes 12, 12′, 12″. Alternatively, in another embodiment, one or more of spokes 12, 12′, 12″ is pivotally joined to the main body of the elongated member 10, for example, by a pin or hinge (not shown). Upon deployment of the elongated member 10 from the distal end 29 of the catheter 28, the hinge or pin (not shown) is activated via the actuator 27 to cause the one or more spokes 12, 12′, 12″ to pivot relative to the other spokes 12, 12′, 12″ at a pivot point 31.

With continued reference to FIG. 2B, in one embodiment, a plurality of wires (not shown) form the elongated member 10. The wires are axially aligned with one another along the proximal portion of the elongated member 10, e.g., collected together in a tube or welded together along the long axis of the wires (not shown). At the pivot point 31, the wires are separated in order to form spokes 12. According to the invention, in one embodiment the number of spokes 12 corresponds to the number of wires that form the elongated member 10. For example, if three spokes 12 are desired, the elongated member 10 will be made of three wires.

Referring to FIG. 2B, the proximal end 80 of the elongated member 10, is in communication with an energy source 42. The energy source 42, in the illustrative embodiment, for example, provides energy to the elongated member 10. The energy delivered to the elongated member 10 may be any form of energy capable of activating the releaseably coupled bonding material 30, for example, by decreasing the viscosity and increasing the flow rate of the bonding material 30 or by increasing the tackiness of the bonding material 30 disposed on the distal end 82 of the elongated member 10 or on the spokes 12 of the elongated member 10. For example, the energy may be radio frequency energy, electrical resistance, ultrasound energy, laser energy, chemical energy, microwave energy, sonic energy, or thermal resistance heating energy.

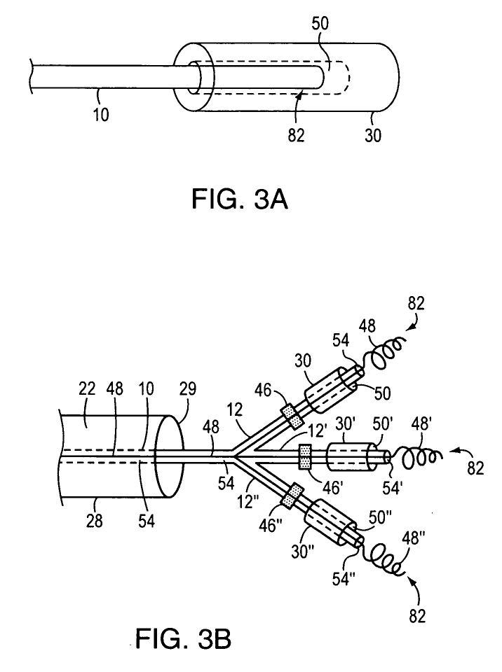

FIG. 3A is a schematic perspective view of a portion of a closure device 14 according to another illustrative embodiment of the invention. According to this illustrative embodiment of the invention, the distal end 82 of the spoke 12 of the elongated member 10 of the closure device 14 includes a releaseably coupled bonding material 30. The bonding material 30 may be in the form, for example, of a sheet that is wrapped around the elongated member 10 or a spoke 12, a tubular sleeve that slides over the elongated member 10 or over a spoke 12, or a coating on the surface of the distal end 82 of the elongated member 10 or a spoke 12. According to this illustrative embodiment, for example, the tubular sleeve of bonding material 30 contains a lumen 50 which allows the tubular sleeve to be slideably disposed over the distal end 82 of the elongated member 10 or spoke 12. For example, in one embodiment, each spoke 12, 12′, 12″ has a tubular sleeve of bonding material 30 that slides over each spoke 12, 12′, 12″. In one embodiment, none of the tubular sleeves of bonding material 30 is connected to any other tubular sleeve of bonding material 30. Alternatively, one or more of the sleeves of bonding material 30 is connected to one other sleeve of bonding material 30. In a preferred embodiment, the releaseably coupled bonding material 30 is energized and deposited in the region of the patent foramen ovale 44 or other cardiac defect 44, more specifically, within the defect 44. Here, the bonding material 30 acts as a framework for endogenous tissue in-growth to encourage permanent closure of the cardiac defect 44 by the patient's own tissue.

FIG. 3B is a schematic perspective view of a portion of another closure device 14 according to another illustrative embodiment of the invention. According to this illustrative embodiment of the invention, the distal end 82 of the elongated member 10 or each spoke 12, 12′, 12″ of the elongated member 10 of the closure device 14 includes a tubular sleeve of releaseably coupled bonding material 30, 30′, 30″. According to this illustrative embodiment, each tubular sleeve of bonding material 30, 30′, 30″ contains a lumen 50, 50′, 50″, respectively, through the full length of the sleeve 30, 30′, 30″ which allows the sleeve of bonding material 30, 30′, 30″ to be slideably disposed over the distal end 82 of the elongated member 10 or of each spoke 12, 12′, 12″.

With continued reference to FIG. 3B, according to this illustrative embodiment of the invention, the elongated member 10 and each spoke 12, 12′, 12″ of the elongated member 10 contains a lumen 54, 54′, 54″, respectively, through which a retractable distal stop 48, 48′, 48″, (collectively 48), is slideably disposed. The distal stop 48, 48′, 48″ may be in the form, for example, of a coil, helix, or other configuration. The distal stop 48 holds the sleeve of bonding material 30, 30′, 30″ in place and prevents the bonding material 30 from moving distally along the elongated member 10. Once the bonding material 30 is appropriately placed in the patent foramen ovale 44, the distal stop 48, 48′, 48″ is slideably retracted or mechanically actuated through the lumen 54, 54′, 54″ of the elongated member 10 or of each spoke 12, 12′, 12″ of the elongated member 10. Additionally, according to this illustrative embodiment, the elongated member 10 and each spoke 12, 12′, 12″ of the elongated member 10 contains a proximal stop 46, 46′, 46″, respectively, that prevents the sleeve of bonding material 30, 30′, 30″ from sliding proximally along the elongated member 10 or spoke 12 prior to delivery, for example, in the patent foramen ovale 44.

With continued reference to FIG. 3B, in one embodiment, the bonding material 30, 30′, 30″ is releaseably adhered to the elongated member 10 or releaseably adhered to the spokes 12 of the elongated member 10. Upon application of energy from the energy source 42, the bonding material 30 releases from the elongated member 10. Until application of energy, the bonding material 30 is adhered to the elongated member 10.

Bonding materials preferably are biocompatible, nontoxic, and degrade into nontoxic components. The bonding material may be, for example, a bioabsorbable polymer including a bioresorbable polymer, a biological material, or a biological material with a bioresorbable or bioabsorbable polymer coating.

Representative bioresorbable or bioabsorbable polymers include, but are not limited to, polylactides, including poly(L-lactides), polycaprolactone, polyglycolides, blends and copolymers thereof.

Representative natural polymers of use as a bonding material in the present invention include, but are not limited to, biological materials, such as a biological tissue scaffold fabricated from, for example, a collagen based material derived from the intestine, stomach, skin, bladder, or pericardium of a porcine animal, a bovine animal, an equine animal and/or a human. Alternatively, the natural polymer may be a protein, such as casein, gelatin, gluten, or serum albumin.

According to a preferred embodiment, the natural polymer is formed of collagen, derived from the tunica submucosa of a porcine small intestine, and delaminated from the other layers of the porcine small intestine by any method known in the art. Alternatively, collagenous tissue from the fascia lata, pericardium, or dura mater of porcine animals or other mammalian sources, such as, for example, cows or sheep, may form the tissue scaffold.

Alternatively, the natural polymer may be formed of one or more polysaccharides, such as cellulose, dextrans, and polyhyaluronic acid, or other biological materials, including but not limited to, deoxyribonucleic acid, ribonucleic acid, and mammalian cells including stem cells, capable of encouraging tissue growth may be used as a bonding material or a component of the bonding material. Furthermore, the biological material may be coated with any of the bioresorbable or bioabsorbable or natural polymers identified above. Moreover, the bonding material may be formed from any combination of the aforementioned materials.

Energy risers, as contemplated by this invention, are portions of the energy delivery closure device 14 adapted to increase the intensity or directionality of the applied energy transmitted by the energy source 42 to the releaseably coupled bonding material 30. Applying energy to a specific location or in a specific intensity or duration to the target intracardiac site allows greater flexibility in the design of an energy delivery closure device 14 or implant. Energy risers allow the operator of the energy delivery closure device 14 greater control over delivery of the energy to the target. For example, an energy delivery closure device 14 including energy risers may allow for greater or smaller quantities of focused energy to be applied, tailoring the energy delivery to the clinical indication and improving the patient outcome.

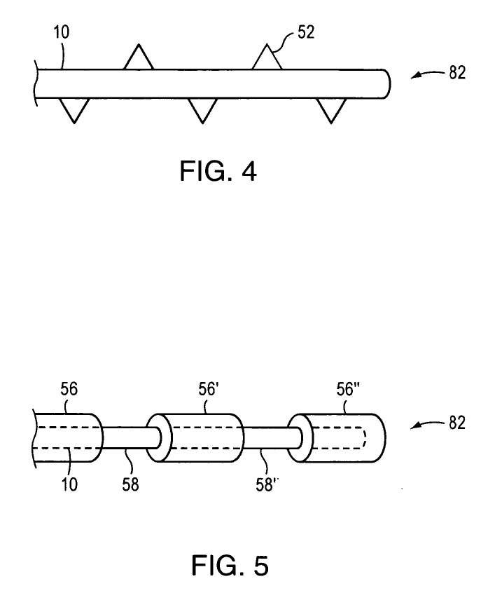

For example, FIG. 4 is a schematic perspective view of a portion of a closure device 14 according to another illustrative embodiment of the invention in which the energy risers are positioned on the elongated member 10 or on the spoke 12 (not shown) in the form of pyramidal extensions 52. The extensions 52, in the illustrative embodiment, for example, may be formed of the same material as the elongated member 10. The closure device 14 may have one or more energy risers, for example, two, three, four, or as many as 100 energy risers.

FIG. 5 is a schematic perspective view of a portion of a closure device 14 according to another illustrative embodiment of the invention in which the energy risers are in the form of at least one, for example, two uninsulated portions 58, 58′, disposed between insulated portions 56, 56′, 56″ of the elongated member 10 or of the spoke 12 (not shown). For example, in one embodiment, a first insulated portion 56 is disposed next to a first uninsulated portion 58. In another embodiment, a first insulated portion 56 is disposed between a first uninsulated portion 58 and a second uninsulated portion 58′. In another embodiment, a first uninsulated portion 58 is disposed between a first insulated portion 56 and a second insulated portion 56′. It is contemplated that the insulating material coating the insulated portions 56, 56′, 56″ may be composed of any material that changes the conductivity properties of the elongated member 10 relative to the energy delivery portions 58, 58′. For example, in one embodiment, the material that changes the conductivity properties of the elongated member 10. In another embodiment, the material is a polymer coating.

Additionally, according to another illustrative embodiment, the energy risers may be in the form of at least one roughed patch (not shown) distributed on the exterior surface of the elongated member 10. For example, in one embodiment, at least a portion of the surface of the elongated member 10 is abraded to roughen the texture of the surface in order to create an energy riser. In a further embodiment, a first roughened patch (not shown) is adjacent to a non-roughened surface (not shown). In yet another embodiment, a first roughened patch (not shown) is disposed between a first non-roughened surface (not shown) and a second non-roughened surface (not shown). In a still further embodiment, a first non-roughened surface (not shown) is disposed between a first roughened patch (not shown) and a second roughened patch (not shown).

Furthermore, the energy risers may be in the form of at least one protuberance (not shown) on the surface of the bonding material 30, a roughening of the surface (not shown) of the bonding material 30, altering the material properties of the bonding material 30, such as a secondary or tertiary process involving coating the bonding material 30 (not shown), and placing alternate materials within a segment of the bonding material 30 (not shown) to sharply change the material properties in the segment relative to the rest of the bonding material 30. For example, in one embodiment, the bonding material 30 includes a material that is activated by energy at a rate different than another material in the bonding material 30. This allows for at least one portion of the bonding material 30 to be activated before another portion of the bonding material 30. In another embodiment, the bonding material 30 includes a portion that is more dense than another portion of the bonding material 30. This allows for one portion to be activated before another portion of the bonding material 30.

Furthermore, it is contemplated that other methods of targeting energy delivery through energy risers may be employed. For example, the cross-sectional geometry of the elongated member 10 or the bonding material 30 may be modified, such as rectangular, square, oval, round, or flat (not shown). Alternatively, the elongated member 10 may be spliced into a plurality of thinner elongated members (not shown), or the material properties of the elongated member 10 or the bonding material 30 may be altered, for example, by performing a secondary or tertiary process involving coating at least a portion of the elongated member 10 or the bonding material 30. For example, in one embodiment, at least a portion of the elongated member 10 is coated with a polymer or a metal. In another embodiment, the material properties of the elongated member 10 are segmentally altered, allowing one segment of the elongated member 10 to have different conductive properties from another segment. For example, in one embodiment, the elongated member 10 includes a first portion of a first density and a second portion of a second density. In another embodiment, the elongated member 10 includes a first portion having a first level of conductivity and a second portion having a second level of conductivity.

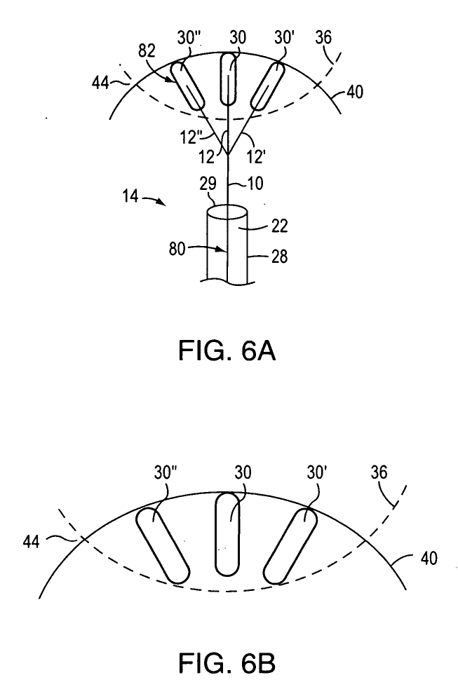

FIGS. 6A and 6B illustrate a series of exemplary steps for a method of closing an intracardiac defect with the closure device 14, according to an illustrative embodiment of the invention. Referring to FIG. 6A, the closure device 14 includes an elongated member 10 slideably disposed in the lumen 22 of the delivery catheter 22, that has been percutaneously and transluminally positioned within the patent foramen ovale 44. The illustrative elongated member 10, for example, includes a plurality of spokes 12, 12′, 12″. Each spoke 12, 12′, 12″ includes a portion of releaseably coupled bonding material 30, 30′, 30″, respectively, in the form of a coating or a slideably disposed sleeve.

With continued reference to FIG. 6A, according to the illustrative embodiment, the spokes 12, 12′, 12″ of the elongated member 10 are inserted past the septum secundum 36 and into the cardiac opening, e.g., the patent foramen ovale 44. The spokes 12, 12′, 12″ are then positioned between the septum secundum 36 and the septum primum 40. The releaseably coupled bonding material 30, 30′, 30″, either coating the spokes 12, 12′, 12″ or in the form of a sleeve slideably disposed over the distal end 82 of the spokes 12, 12′, 12″, is also positioned between the septum secundum 36 and the septum primum 40 of the cardiac opening.

Still referring to FIG. 6A, when energy transmitted from the energy source 42 (not shown) is applied to the elongated member 10, the energy is transferred, at least in part, to the releaseably coupled bonding material 30. For example, when the energy is applied, the tackiness of the bonding material 30 increases and/or the flow rate of the energized bonding material 30 increases and the viscosity of the bonding material 30 decreases.

With continued reference to FIG. 6A, according to one illustrative embodiment of the invention, the energized bonding material 30, for example, is released from the elongated member 10 and associates with the tissue of the septum secundum 36 and the septum primum 40. Following association of the bonding material 30 to the tissue, the elongated member 10 is retracted from the intracardiac opening, leaving the bonding material 30 behind (as depicted in FIG. 6B) in the intracardiac defect 44.

Still referring to FIG. 6A, according to another illustrative embodiment of the invention, the proximal end 80 of the elongated member 10 or the spoke 12 contains a proximal stop 46 (not shown) and the distal end 82 of the elongated member 10 or the spoke 12 contains a retractable distal stop mechanism 48 (not shown). According to this embodiment, the distal stop mechanism 48 (not shown) is reversibly attached to the sleeve of releaseably coupled bonding material 30, for example, by a hook and loop, ball and socket, claw, screw, or other reversible attachment mechanism. Furthermore, the elongated member 10 may be in the form of a hollow tube (not shown), with the retractable distal stop mechanism slideably disposed within the lumen 54 (not shown) of the hollow tube elongated member 10.

With continued reference to FIG. 6A, according to one exemplary embodiment of the invention, the elongated member 10 or the spoke 12 containing the releaseably coupled bonding material 30 is inserted between the septum secundum 36 and the septum primum 40 of the patent foramen ovale 44, energy is applied to the elongated member 10, and the energized bonding material 30 associates with the tissue of the septum secundum 36 and the septum primum 40. Following association of the bonding material 30 to the tissue, the distal stop mechanism 48 (not shown), for example, is disengaged from the bonding material 30, retracted into the hollow tube of the exemplary elongated member 10, and the elongated member 10 retracted from the intracardiac opening, leaving the bonding material 30 behind in the cardiac defect 44 (as depicted in FIG. 6B).

FIG. 6B illustrates a top schematic perspective view of a portion of a closure device 14 including a closed intracardiac opening according to an illustrative embodiment of the invention. FIG. 6B illustrates the positioning of the energized bonding material 30 after it is released from the elongated member 10. The bonding material 30, 30′, 30″ is placed between the septum secundum 36 and the septum primum 40 to encourage tissue in-growth and closure of the intracardiac opening.

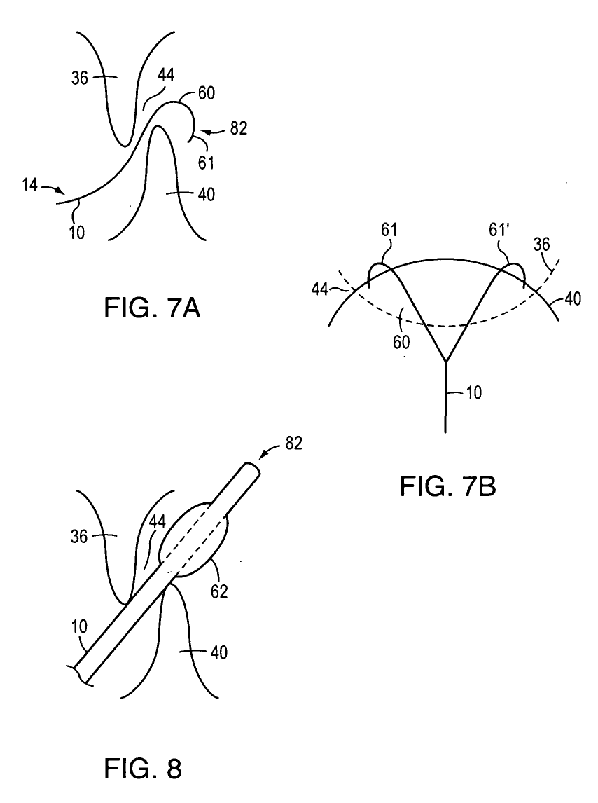

Optionally, as illustrated in FIGS. 7A, 7B, and 8, the closure device 14 (not shown) further includes a locator 60, 62 (generally 60). The locator 60 may be connected to the elongated member 10 or either to or within the delivery catheter 28 (not shown). In one embodiment, the locator 60 is integral to the elongated member 10, and is disposed at the distal end 82 of the elongated member 10. In another embodiment, the locator 60 is maintained within a collapsed state within the lumen 54 of the elongated member 10, and is deployed to an open state beyond the distal end 82 of the elongated member 10 in order to position the elongated member 10 in the patent foramen ovale 44. In one embodiment, the physician positions the locator 60 between the septum secundum 36 and the septum primum 40 of the patient's heart. The locator 60 is used by the physician, for example, to limit movement of the septum secundum 36 and of the septum primum 40 prior to positioning, as explained above, the elongated member 10 and the bonding material 30 within the patent foramen ovale 44. The locator 60 also serves to position the distal end (not shown) of the delivery catheter 28 (not shown) in the area where the septum secundum 36 and the septum primum 40 overlap.

Exemplary locator devices, including flexible members suitable for stabilizing cardiac tissues in a patient and for placing the elements described above in the area where the septum secundum 36 and the septum primum 40 overlap include those described below and those described in U.S. patent application Ser. No. 10/660,444, filed Sep. 11, 2003, and published on May 13, 2004, as U.S. Patent Application Publication No. 2004-0092973, which is incorporated herein by reference in its entirety. For example, the locator 60 may: i) be a flexible coil having a spiral shape, ii) include three flexible hexagonal members forming, generally, a planar array, iii) include two flexible members, each one of which includes a leg, such as a wire, that is pre-shaped to articulate one or more times upon exit from a lumen, iv) include two flexible members, each one of which includes a loop section, or v) be a single flexible member that forms a closed loop.

In the illustrative embodiment depicted in FIG. 7A, the locator 60 is in the form of a hook. The locator 60 may be inserted into the cardiac defect 44, for example, by inserting the locator 66 past the septum secundum 36 and over the septum primum 40, through the patent foramen ovale 44. As shown in FIG. 7A, the hook 60 may be configured such that the distal end 82 of the hook locator 60 temporarily overhangs the septum primum 40 and maintains the closure device 14 in the correct orientation between the septum secundum 36 and the septum primum 40 during energy delivery and release of the bonding material 30 (not shown) in the patent foramen ovale 44. The locator 60 may be either distal or proximal to the site of energy delivery on the elongated member 10.

FIG. 7B is a top schematic perspective view of a portion of the locator 60 of FIG. 7A according to another illustrative embodiment of the invention. In the illustrative embodiment, the elongated member 10 includes a locator 60 with two hooks 61, 61′. In a further embodiment, the elongated member 10 includes a locator 60 with only one hook 61. In yet another embodiment, there may be a plurality of hooks 61, such as three, four, five or more hooks 61. In one embodiment, the locator 60 includes a bonding material 30, while in another embodiment, the locator does not include a bonding material 30. In one embodiment, the locator 60 conducts and delivers energy to the tissue contacted by the locator. In another embodiment, the locator 60 or portions of the locator 60 are insulated such that energy is not transmitted to the contacted tissue through the insulated portions of the locator 60.

FIG. 8 is a side schematic view of a portion of the closure device 14 including a balloon locator 62 positioned in a patent foramen ovale 44 according to another illustrative embodiment of the invention. According to this illustrative embodiment, the elongated member 10 includes, for example, an inflatable balloon 62 portion near or substantially positioned at the distal end 82 of the elongated member. For example, in one embodiment, the balloon 62 is positioned on the surface at the distal end 82 of the elongated member 10. In another embodiment, the balloon locator 62 is maintained within the lumen 54 of the elongated member 10 until it is deployed to locate the patent foramen ovale 44.

The balloon locator 62 may be inserted into the cardiac opening such that the distal end 82 of the elongated member 10 and the balloon locator 62 portion of the elongated member 10 pass over the septum secundum 36 and then the septum primum 40. Following insertion past the septum primum 40, the balloon locator 62 may be inflated using, for example, saline. The elongated member 10 may then be retracted until the balloon locator 62 is located at the distal surface of the septum primum 40. In this configuration, the balloon locator 62 maintains the closure device 14 in the correct orientation between the septum secundum 36 and the septum primum 40 during energy delivery and release of the bonding material 30 (not shown) in the patent foramen ovale 44. In one embodiment of the invention, the balloon locator 62 may or may not include bonding material 30 (not shown). In another embodiment, the balloon locator 62 is located proximal to the bonding material 30. Furthermore, in another embodiment the elongated member 10 containing the balloon locator 62 conducts and delivers energy to the tissue which it contacts, while in another embodiment, the balloon locator 62 or a portion or portions of the balloon locator 62 are insulated to inhibit energy delivery by the balloon locator 62. In all embodiments, it is contemplated that the locator 60, 62 is removed after delivery of energy to the target site.

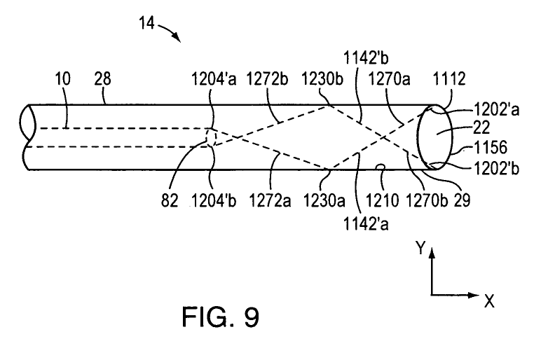

FIG. 9 illustrates a portion of a closure device 14 according to an illustrative embodiment of the invention including exemplary flexible members 1142′a and 1142′b for positioning the closure device 14 in an intracardiac defect 44. In one embodiment, the flexible members 1142′a and 1142′b are disposed at the distal end 82 of the elongated member 10, distal to the bonding material 30 (not shown). In another embodiment, the flexible members 1142′a and 1142b are disposed at the distal end 82 of the elongated member 10, for example, at the distal end of a spoke 12. Each of the flexible members 1142′a and 1142′b include a leg such as a wire having a first end 1204′a and 1204′b, respectively, joined to the distal end 82 of the elongated member 10. Each of the flexible members 1142′a and 1142′b also have a second distal end 1202′a and 1202′b, respectively, that is free, i.e., not joined to any other structure of the closure device 14. The longitudinal axis of the flexible members 1142′a and 1142′b are oriented substantially parallel to the delivery catheter 28 when the flexible members 1142′a and 1142′b are located within the lumen 22 of the delivery catheter 28. The flexible members 1142′a and 1142′b have a first portion 1272a and 1272b, respectively and a second portion 1270a and 1270b, respectively. The flexible members 1142′a and 1142b are disposed within the lumen 22 in this contracted position such that the second ends 1202′a and 1202′b are directed distally 82 towards the opening 1112 in the distal end 29 of the delivery catheter 28.

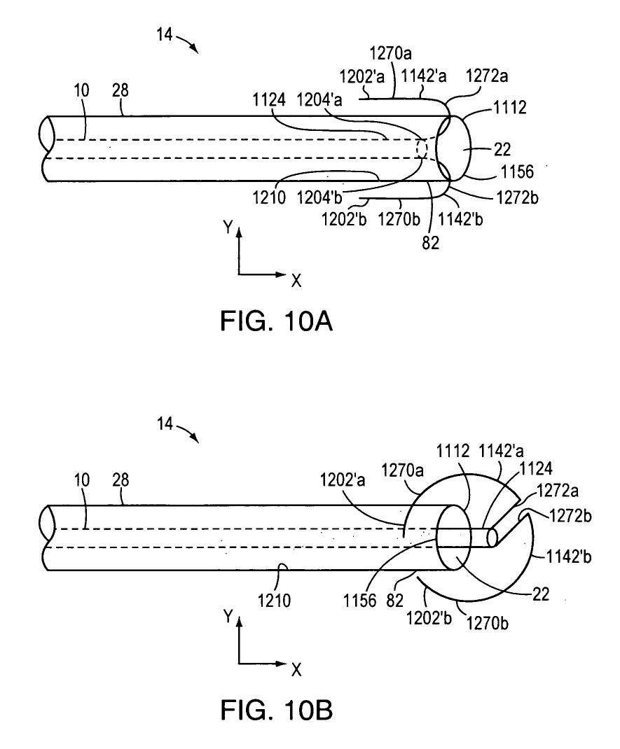

Prior to insertion into the lumen 22, the flexible members 1142′a and 1142′b are preshaped such that the flexible members 1142′a and 1142b will assume a predetermined extended configuration when the flexible members 1142′a and 1142′b are free from the confines of the lumen 22. The flexible members 1142′a and 1142b are freed from the confines of the lumen 22 by moving the flexible members 1142′a and 1142′b between the contracted position illustrated, for example, in FIG. 9 and an extended position, such as the extended position depicted in FIG. 10B. While in the lumen 22 of the delivery catheter 28, the flexible members 1142′a and 1142′b apply a force to an inner surface 1210 of the delivery catheter 28 in a first location 1230a and 1230b, respectively, on the inner surface 1210 of the lumen 22 that the flexible members 1142′a and 1142b contact. The force applied by the preshaped flexible members 1142′a and 1142′b to the inner surface 1210 is the resultant force associated with the inner surface 1210 constraining the shape of the flexible members 1142′a and 1142b so they may fit within the lumen 22 of the delivery catheter 28.

In an embodiment of a closure device referring now to FIG. 10A, the flexible members 1142′a and 1142′b are shown partially extended (in comparison with the flexible members 1142′a and 1142′b in FIG. 9) so the flexible members 1142′a and 1142b are still substantially parallel to the longitudinal axis of the delivery catheter 28. As the elongated member 10 is extended out of the opening 1112 of the delivery catheter 28, the second ends 1202′a and 1202′b of the flexible members 1142′a and 1142′b, respectively, undergo an articulation and point, generally, in a proximal direction toward the handle (not shown). In this orientation, the preshaped flexible members 1142′a and 1142′b apply a force to the rim 1156 of the opening 1112 in the delivery catheter 28 because the rim 1156 of the opening 1112 constrains the shape of the flexible members 1142′a and 1142′b.

The elongated member 10 is further extended distally, referring now to FIG. 10B, along the lengthwise dimension (in the positive direction along the X-axis) of the lumen 22 until the distal end 82 of the elongated member 10 emerges from the opening 1112 of the delivery catheter 28. The second ends 1202′a and 1202′b of the exemplary preshaped flexible members 1142′a and 1142′b, respectively, undergo an additional articulation and as a result point, generally, towards one another. In this extended position, the preshaped flexible members 1142′a and 1142′b no longer apply a force to the delivery catheter 28 because the delivery catheter 28 does not constrain the shape of the flexible members 1142′a and 1142b. In this extended position, each of the flexible members 1142′a and 1142′b is substantially planar in shape. The plane of each of the flexible members 1142′a and 1142′b define a plurality of axes that lie in the plane. The plurality of axes are non-parallel to (i.e., biased relative to) the longitudinal axis of the delivery catheter 28. For example, the plurality of axes defined by the planes of the flexible members 1142′a and 1142′b are positioned at an angle in the range of about 0 degrees to about 180 degrees, preferably, about 90 degrees, relative to the longitudinal axis of the elongate member 10.

In alternative embodiments of the invention, the second ends, for example, the second ends 1202′a and 1202′b, may have a different diameter than other locations along the length of the flexible elastic members 1142′a and 1142′b. By way of example, an operator may select an apparatus having flexible members that have second ends 1202′a and 1202′b having a larger diameter to, for example, reduce trauma to tissue the second ends 1202′a and 1202′b contact during use. Alternatively, the second ends 1202′a and 1202′b may have a ball shaped tip.

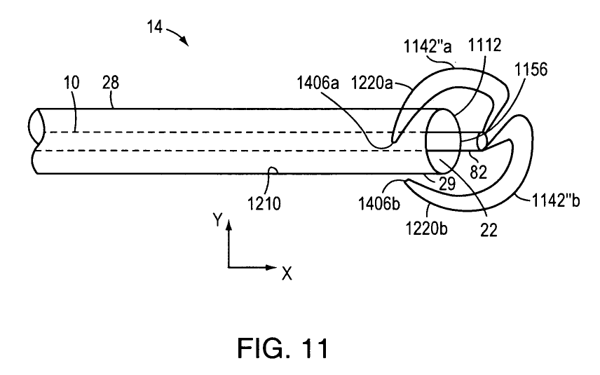

FIG. 11 depicts a portion of a closure device 14 including flexible members for positioning the closure device at an intracardiac defect according to an alternative illustrative embodiment of the invention. The exemplary flexible members 1142″a and 1142″b include a first wire loop section 1220a and a second loop section 1220b, respectively, as illustrated in FIG. 11. The tip 1406a and 1406b of the loop sections 1220a and 1220b, respectively, point, generally, towards one another and towards the elongated member 10. Loop sections 1220a and 1220b may, alternatively, be oriented in a variety of directions (e.g., away from the elongated member 10 or at a 45 degree angle away from the elongated member 10). However, the loops 1220a and 1220b of the flexible members 1142″a and 1142″b will, typically, be oriented such that the flexible members 1142″a and 1142″b including the loop sections 1220a and 1220b are substantially planar, where the plane defines a plurality of axes lying in the plane. The plurality of axes are non-parallel to (i.e., biased relative to) the longitudinal axis of the delivery catheter 28 when the flexible members 1142″a and 1142″b are extended distally from the opening 1112 of the delivery catheter 28. Other embodiments of the flexible member 1142″a and 1142″b are also contemplated by the invention and are not limited to those illustrated.

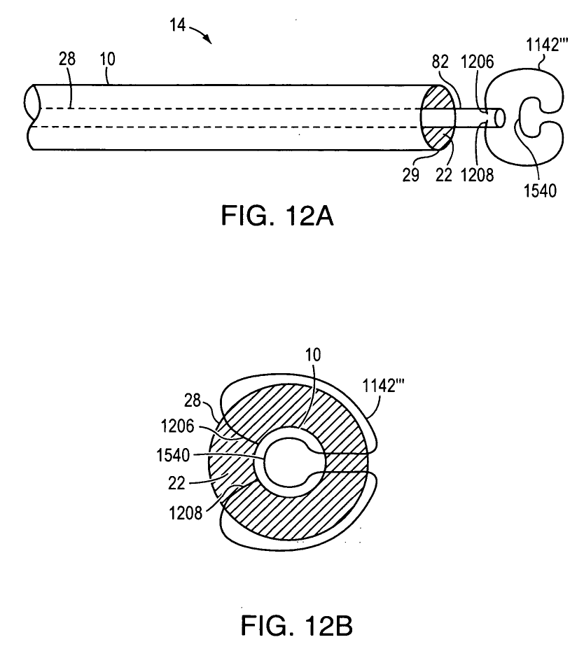

In an alternative embodiment, referring now to FIGS. 12A and 12B, the closure device 14 includes a single flexible member 1142′″ for positioning the closure device in an intracardiac defect 44, such as a patent foramen ovale. The flexible member 1142′″ is disposed at the distal end 82 of the elongated member 10, distal to the bonding material 30 according to one embodiment of the invention. The flexible member 1142′″ has a first end 1206 and a second end 1208; both the first end 1206 and the second end 1208 are connected to the distal end 82 of the elongated member 10. The flexible member 1142′″ also has a middle section 1540 located, generally, intermediate the first end 1206 and the second end 1208 of the flexible member 1142′″. The flexible member 1142′″ thereby forms a closed loop. In this embodiment, the flexible member 1142′″ is configured so the middle section 1540 is located, generally in the center of a plane defined by the flexible member 1142′″ as illustrated by the end-on view of FIG. 12B. In this configuration, the middle section 1540 of the flexible member 1142′″ aids with stiffening the flexible member 1142′″ as compared with the embodiment of the invention illustrated in FIGS. 10A and 10B where the flexible members 1142′a and 1142b have free ends 1202′a and 1202′b, respectively. The stiffening minimizes bending when, for example, the flexible member 1142′″ is used by an operator to apply forces to a tissue, e.g., the atrial septum. In this configuration, the flexible member 1142 forms a closed loop that is sized and shaped, for example, to contact a first and second side of a tissue, similarly, as described herein.

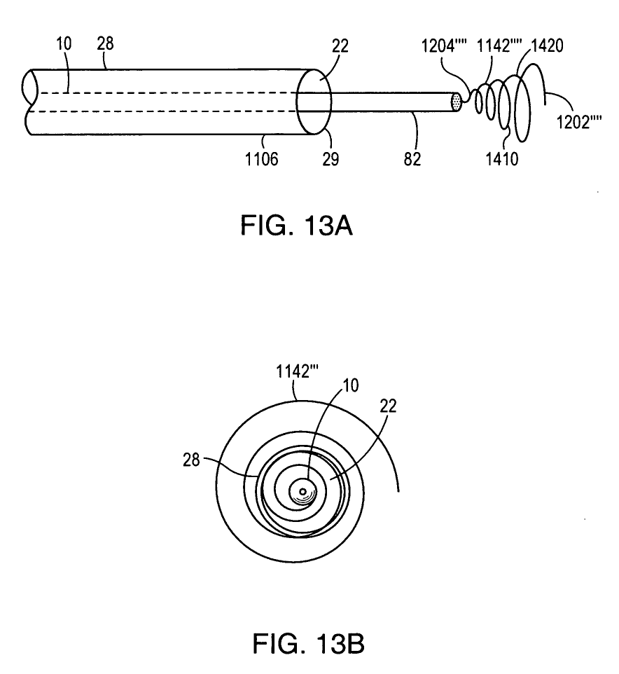

In another embodiment of the invention, referring now to FIGS. 13A and 13B, the flexible elastic member 1142″″ is a coil (coil-like member) and has a spiral shape extending from a narrow first end 1204″″ to a broad second end 1202″″. The flexible elastic member 1142″″ assists in the positioning of the closure device 14 at the site of the intracardiac defect 44. The narrow first end 1204″″ is connected to the distal end 82 of the elongated member 10. Referring now to FIG. 13B, the flexible member 1142″″ is oriented such that one spiral of the flexible member substantially defines a plane. The plane defines a plurality of axes lying in the plane and the plurality of axes are non-parallel to the longitudinal axis of the delivery catheter 28. When the elongated member 10 is withdrawn into the lumen 22 of the delivery catheter 28, the flexible member 1142″″ substantially parallels the longitudinal axis of the delivery catheter 28. By way of example, in use, a portion 1410 of the flexible member 1142″″ can be located on a first side of a tissue and a portion 1420 of the flexible member 1142″″ can be located on a second side of a tissue. For example, the flexible member 1142″″ can be screwed through a tunnel or a hole, such as a defect in the atrial septum. Alternatively, the distal end 82 of the elongated member 10 may be located axially through, for example, a hole in a tissue such that the flexible member 1142″″ may be withdrawn partially through the hole by a rotational (screw-like) motion of the elongated member 10 thereby locating the portion 1410 of the flexible member 1142″″ on a first side of the tissue and the portion 1420 of the flexible member 1142″″ on a second side of a tissue.

In alternative embodiments of the spiral shaped flexible elastic member 1142″″, the spiral can, for example, extend from a broad first end 1204″″ to a narrow second end 1202″″, have a substantially equal diameter along the length of the spiral flexible elastic member 1142″″ along the longitudinal axis of the delivery catheter 28, or vary in diameter along the length of the spiral flexible elastic member 1142″″ along the longitudinal axis of the delivery catheter 28. The shape of the spiral and or parts thereof can also, for example, be chosen to approximate or match the geometry of the defect.

In one embodiment of a spiral shaped device, the flexible elastic member 1142″″ has the spacing between sections of the spiral that varies in relation to the longitudinal axis of the delivery catheter 28. By way of example, the spacing between sections of the spiral at the first end 1204″″ is about 1.0 mm and decreases in a linear fashion to a spacing of about 0.25 mm between sections of the spiral at the second end 1202″″. An operator might select the spacing between sections of the spiral that, for example, approximates the thickness of a tissue.

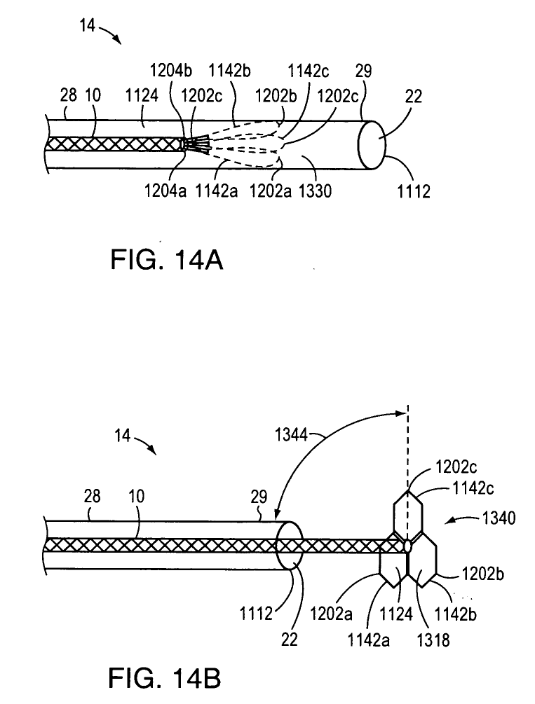

In an alternative embodiment of the present invention, as illustrated in FIGS. 14A and 14B, first ends 1204a, 1204b, and 1204c of the exemplary three flexible members 1142a, 1142b, and 1142c, respectively, are connected to a distal end 82 of the elongated member 10. The flexible member 1142 assist in positioning the closure device 14 at the site of the intracardiac defect 44.

Referring now to FIG. 14B, the elongated member 10 and the exemplary flexible members 1142a, 1142b, and 1142c are initially collapsed within the lumen 22 of delivery catheter 28 in a contracted first position 1330. The contracted flexible members 1142a, 1142b, and 1142c are disposed within the lumen 22 of the delivery catheter 28 such that the flexible members 1142a, 1142b, and 1142c lie substantially parallel to the longitudinal axis of the lumen 22 of the delivery catheter 28.

In this embodiment of the invention, the elongated member 10 is translated axially along the lengthwise dimension of the lumen 22 until the distal end 82 of the elongated member 10 emerges from an opening 1112 at the distal end 29 of the delivery catheter 28 and the flexible members 1142a, 1142b, and 1142c transition from the contracted first position 1330 shown in FIG. 14A to a second extended position 1340 shown in FIG. 14B. The exemplary flexible members 1142a, 1142b, and 1142c expand to assume, for example, substantially hexagonal shapes upon emerging from the opening 1112 in the delivery catheter 82 and expanding. The extended flexible members 1142a, 1142b, and 1142c are substantially planar. The plane defines a plurality of axes that lie in the plane and the plurality of axes are non-parallel to (i.e., biased relative to) the delivery catheter 28. An angle 1344 defined by at least one of the plurality of axes of the plane of the flexible members 1142a, 1142b, and 1142c and the longitudinal axis of the delivery catheter 28 can be between about 0 degrees and about 180 degrees. The angle 1344 is typically specified (e.g., by an operator) such that the flexible members 1142a, 1142b, and 1142c are flush with tissue surface and are capable of applying a force across a large tissue area. For example, the angle 1344 might be chosen to ensure the flexible members 1142a, 1142b, and 1142c conform to the shape of a tissue surface abutting the flexible members 1142a, 1142b, and 1142c. If the force is applied, e.g., across a large tissue area the movement of the tissue in any location across the tissue area will be minimized. The flexible members 1142a, 1142b, and 1142c could, alternatively, be of any shape (e.g., polygonal, circular, or ellipsoidal) or of any quantity (e.g., one, two, or five) where the shape and/or quantity of the flexible members 1142a, 1142b, and 1142c are typically selected to distribute as much force as possible while still being able to fit within the lumen 22 of the delivery catheter 28 and emerge from or retract into the lumen 22.

Variations, modifications, and other implementations of what is described herein will occur to those of ordinary skill in the art without departing from the spirit and the scope of the invention as claimed. Accordingly, the invention is not to be defined by the preceding illustrative description but instead by the spirit and scope of the following claims.

What we claim is:

1. A system for percutaneous transluminal closure of an intra-cardiac opening, comprising:
a catheter comprising a lumen axially disposed between a proximal end and a distal end;
an elongated member slideably disposed in the lumen of said catheter, said elongated member having a proximal end and a distal end, said elongated member having a plurality of spokes at the distal end; and
an energy activatable bonding material positioned on the outer surface of each of said plurality of spokes.

2. The system of claim 1, further comprising an energy source operatively connected to the proximal end of said elongated member wherein said elongated member transfers energy to said bonding material.

3. The system of claim 1, wherein said bonding material comprises a sleeve comprising a lumen wherein the distal end of said elongated member is slideably disposed in the lumen of said bonding material sleeve.

4. The system of claim 1, wherein said bonding material comprises a coating releaseably adhered to the distal end of said elongated member, said coating being releasable upon application of energy to said coating.

5. The system of claim 3, wherein said elongated member further comprises a lumen wherein a retractable distal stop is slideably disposed in the lumen of said elongated member.

6. The system of claim 1, wherein the bonding material comprises a bioabsorbable material.

7. The system of claim 6, wherein the bioabsorbable material is selected from the group consisting of poly-L-lactic acid, polylactic acid, polyglycolic acid, and copolymers and combinations thereof.

8. The system of claim 1, wherein the bonding material comprises a biological material.

9. The system of claim 8, wherein the biological material comprises a composition selected from the group consisting of collagen, cellulose, and the intestinal collagen layer.

10. The system of claim 9, wherein the intestinal collagen layer comprises tunica submucosa of a porcine small intestine.

11. The system of claim 1, wherein the bonding material comprises a synthetic material.

12. The system of claim 11, wherein the synthetic material comprises a polymer.

13. The system of claim 1, further comprising an energy riser selected from the group consisting of a protuberance on the surface of the elongated member, a noninsulated portion of the elongated member disposed between two insulated portions of the elongated member, a roughened surface of the elongated member, an elongated member comprising a plurality of materials, and placing alternate materials within a segment of the elongated member to sharply change the material properties in the segment relative to the rest of the elongated member.

14. The system of claim 1, further comprising an energy riser selected from the group consisting of a protuberance on the surface of the bonding material, a roughened surface of the bonding material, changing the material properties of the bonding material, and placing alternate materials within a segment of the bonding material to sharply change the material properties in the segment relative to the rest of the bonding material.

15. The system of claim 1, wherein the energy source is selected from the group consisting of radio frequency energy, electrical resistance, ultrasound energy, laser energy, chemical energy, microwave energy, sonic energy, and thermal resistance heating energy.

16. The system of claim 1, wherein the elongated member comprises a non-insulated portion.

17. The system of claim 1, wherein the cross-section of the elongated member is non-circular.

18. The system of claim 1, further comprising a first locator distal to said energy source, wherein said first locator positions said elongated member in said cardiac opening.

19. The system of claim 1, further comprising a second locator proximal to said energy source, wherein second locator positions said elongated member in said cardiac opening.

20. The system of claim 18, wherein the locator comprises a balloon.

21. The system of claim 18, wherein the locator comprises a hook.

22. The system of claim 18, wherein the locator is insulated.

23. A system for percutaneous transluminal closure of an intra-cardiac opening, comprising:
a catheter comprising a lumen axially disposed between a proximal end and a distal end;
an elongated member slideably disposed in the lumen of said catheter, said elongated member having a proximal end and distal end, said elongated member having a plurality of spokes at the distal end; and
an energy riser disposed on said elongated member.

24. The system of claim 23, wherein the energy riser is selected from the group consisting of a protuberance on the surface of the elongated member, a noninsulated portion of the elongated member disposed between two insulated portions of the elongated member, a roughened surface of the elongated member, changing the elongated member material properties, and placing alternate materials within a segment of the elongated member to sharply change the material properties in the segment relative to the rest of the elongated member.

25. The system of claim 23, further comprising a bonding material disposed on said plurality of spokes.

26. The system of claim 23, further comprising a locator.

27. A method for percutaneous transluminal closure of an intracardiac opening, comprising the steps:
a) inserting a catheter comprising a locator and an elongated member having a plurality of spokes into a patient;
b) locating a patent foramen ovale with the locator;
c) positioning the elongated member comprising at least one sleeve of bonding material;
d) applying energy to the elongated member;
e) adhering the bonding material to the intracardiac opening; and
f) removing the elongated member and locator.
